(19) United States
(12) Patent Application Publication
(10) Pub. No.: US 2019/0307357 A1
(43) Pub. Date: Oct. 10, 2019

(54) ELECTRICALLY RESONANT ELECTRODE CONFIGURATION FOR MONITORING OF A TISSUE

(71) Applicant: Brigham Young University, Provo, UT (US)

(72) Inventors: Kim Manwaring, Phoenix, AZ (US); Preston Manwaring, Farmington, UT (US)

(21) Appl. No.: 16/156,888

(22) Filed: Oct. 10, 2018

Related U.S. Application Data
(63) Continuation of application No. 13/928,279, filed on Jun. 26, 2013, now abandoned.
(60) Provisional application No. 61/665,130, filed on Jun. 27, 2012.

(51) Int. Cl. A61B 5/05; A61B 5/042

(52) U.S. Cl. CPC A61B 5/05; A61B 5/6868; A61B 5/042

(57) ABSTRACT

Electrical impedance monitoring of a tissue or an organ for perfusion or viability has been limited by sensitivity and baseline shifts. An apparatus and method are described which improve sensitivity by making the intervening tissue between pairs of electrodes a determinant component of electrical resonance. Such sensitivity further enhances detection of the pulsatile component of blood flow within a tissue. Baseline shift can be monitored and compensated due to resonance shift. The method is adaptable to sufficiency of perfusion monitoring or viability, imaging by 2-dimensional or 3-dimensional electrical impedance tomography, monitoring of tissue ablation by thermal or chemical methods, and thermoplasty of tissues to alter their form and functionality.

### BACKGROUND OF THE INVENTION

### 1. The Field of the Invention

The present invention relates generally to monitoring tissues and organs. More specifically, the present invention relates to an electrode configuration and method for monitoring tissues and organs.

### 2. State of the Art

The electrical properties of internal organs and tissues may be monitored to provide doctors and caregivers with the condition of the internal organs. Conventional electrical impedance monitoring or tomography (EIT) is based on measurement of current or impedance between pairs of electrodes. A previous patent application by the current inventors, US Pub. No. 2010/021095, describes an electrode configuration and method for impedance monitoring of a tissue based on the use of a central electrode. The application describes specifically an adaptation to monitor brain perfusion and compliance with reconstructed images or electrical impedance tomography (EIT), and it further describes adaptations for use with other tissues such as fetal monitoring (where the central electrode might be a vaginal-positioned electrode below the cervix), transthoracic imaging (where the electrode could be positioned upon an endotracheal tube), and a similar adaptation for carotid artery perfusional symmetry.

A limitation of these methods may be the signal to noise ratio in an electrically noisy environment of the monitored patient or by motion of the patient, such as might be encountered in an intensive care unit or surgical suite. A further limitation may be the known gradual baseline shift in impedance between electrode pairs which limits usability and interpretation over many hours of observation. While multi-factorial, such a shift, may be at least attributable to ionic compositional shift of tissues and perfusing blood, alteration of impedance due to the electrode-tissue interface, and the underlying changes of injury in the organ (for example edema and inflammation). Thus there is a need to improve the method and devices for monitoring tissues and organs to reduce the signal to noise ratio and reduce shifting of the baseline in order to obtain more accurate measurements.

### SUMMARY OF THE INVENTION

According to one aspect of the present disclosure, an electrode configuration is provided wherein electrodes may be placed such that the internal tissue to be monitored is between pairs of electrodes, and such that the internal tissue may be a component of electrical resonance.

According to one configuration, a resonant circuit may include a frequency generator. The resonant circuit may also include one or more of the following: an auto tuner, a coax line, and a balun.

According to one aspect of the present disclosure, detection of the pulsatile component of blood flow within a tissue may be enhanced due to sensitivity of the resonant circuit. Baseline shift may also be monitored and compensated due to resonance shift.

According to a method of the present disclosure, an electrode, hereinafter referred to as a central electrode or internal electrode may be placed within a cavity, such as a natural orifice or a physician-created pathway, and a second electrode may be placed externally. The tissue between the central or internal electrode and the secondary electrode may be a determinant component of the resonant circuit.

According to another aspect of the present disclosure, a central electrode may be used in conjunction with a multiplicity or array of secondary electrodes.

According to another aspect of the present disclosure, there may be numerous applications of the principles of the electrode configuration and resonant circuit as described herein. For example, the method may be adaptable to sufficiency of perfusion monitoring or viability, imaging by 2-dimensional or 3-dimensional electrical impedance tomography, monitoring of tissue ablation by thermal or chemical methods, and thermoplasty of tissues to alter their form and functionality.

These and other aspects of the present invention are realized in an electrode configuration and method of use as shown and described in the following figures and related description. It will be appreciated that various embodiments of the invention may not include each aspect set forth above and aspects discussed above shall not be read into the claims unless specifically described therein.

### BRIEF DESCRIPTION OF THE DRAWINGS

Various embodiments of the present disclosure are shown and described in reference to the numbered drawings wherein:

FIG. 1 shows a diagram of an electrode configuration according to the present disclosure;

FIG. 2 shows a front view of an intracranial bolt which may be used as a central electrode in the electrode configuration as described herein;

FIG. 3A shows a graphical representation of an arterial pressure pulse and the return power or reflected component of standing wave ratio derived from the brain, as well as its cross-correlation;

FIG. 3B shows a graphical representation of plots from a vector analyzer of standing wave ratio, theta, return loss RL, and real component Rs of FIG. 3A;

FIG. 4 shows a sagittal cross-sectional view of a gravid uterus with a possible application of the present disclosure to monitor fetal heart rate;

FIG. 5A shows a graphical representation of derived impedance as return power or reflected component of the measured standing wave ratio (SWR) from a pregnant mother using the electrode configuration of FIG. 4;

FIG. 5B shows a graphical representation of a Fast Fourier Transform (FFT) of the RP of the SWR component shown in FIG. 5A; and

FIG. 6 shows a cross-sectional tomogram of tissue lesioning by pH shift utilizing an electrode configuration as described herein.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The skilled artisan will understand, however, that the methods described below can be practiced without employing these specific details, or that they can be used for purposes other than those described herein. Indeed, they can be modified and can be used in conjunction with products and techniques known to those of skill in the art in light of the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one embodiment,” “one configuration,” “an embodiment,” or “a configuration” means that a particular feature, structure, or characteristic described in connection with the embodiment may be included in at least one embodiment, etc. The appearances of the phrase “in one embodiment” in various places may not necessarily limit the inclusion of a particular element of the invention to a single embodiment, rather the element may be included in other or all embodiments discussed herein.

Furthermore, the described features, structures, or characteristics of embodiments of the present disclosure may be combined in any suitable manner in one or more embodiments. In the following description, numerous specific details are provided, such as examples of products or manufacturing techniques that may be used, to provide a thorough understanding of embodiments of the invention. One skilled in the relevant art will recognize, however, that embodiments discussed in the disclosure may be practiced without one or more of the specific details, or with other methods, components, materials, and so forth. In other instances, well-known structures, materials, or operations may not be shown or described in detail to avoid obscuring aspects of the invention.

Before the present invention is disclosed and described in detail, it should be understood that the present invention is not limited to any particular structures, process steps, or materials discussed or disclosed herein, but is extended to include equivalents thereof as would be recognized by those of ordinarily skill in the relevant art. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “a tissue” may include an embodiment having one or more of such tissues, and reference to “the layer” may include reference to one or more of such layers.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing the nearly all of the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

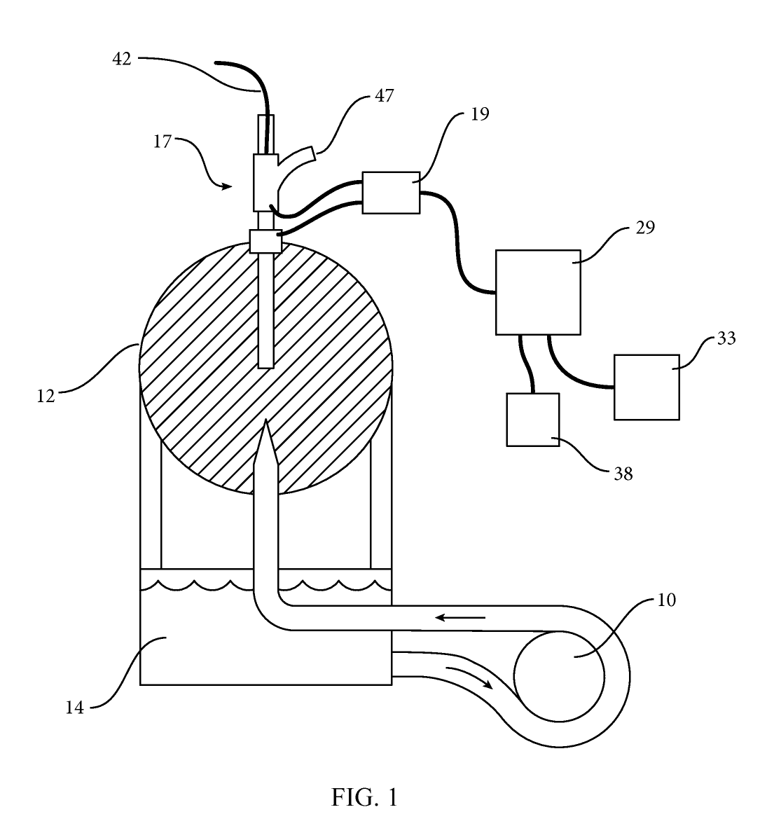

Turning now to FIG. 1, there is shown a layout of an apparatus for resonant tissue monitoring where the tissue is simulated on the bench by a perfusable foam ball. An inherent amplification effect enhancing perfusional sensitivity may be achieved by using the intervening tissue between electrode pairs as the changing component of a resonant circuit, with all other components of the circuit fixed or re-tunable to recover resonance. The baseline frequency for such resonance can be seen to shift as the tissue changes due to causes as cited above. Re-tuning to resonance can be performed by changing componentry values in the circuitry. However, if no components are changed, the frequency of resonance becomes a monitorable variable indicative of tissue change.

Further, the systolic-diastolic aspects of tissue perfusion are amplified by a sensitive circuit with high Q factor (quality factor). The resulting waveform is felt representative of the beat-to-beat effect of pulsatile perfusion or organ volume change due to blood flow. This waveform can be compared to the pressure waveform, EKG, or other systemic waveforms, e.g. oximetry, for changes in lag time and amplitude.

A central or internal electrode may be defined as an electrical contact point within the substance of an organ, tissue, or body utilizing either a natural orifice (i.e., the oropharynix, esophagus, trachea, urethra, rectum, vagina, etc.) or a created access, (i.e., placement of external ventricular drain in the brain, arterial intraluminal catheter by arterial puncture, central venous catheter by venipuncture, radiographic positioning, etc.). A second electrode may be positioned within a tissue, or alternatively may be placed upon or beyond a tissue through which an interrogative current may be passed. A multitude of electrodes may be similarly deployed to allow cross-sectional or even 3-dimensional monitoring by sequential or simultaneous derivation of resonance frequencies.

According to FIG. 1, there is shown a diagram of a simulation of how the present disclosure may be used in monitoring a patient's cranium. The tissue may be simulated on the bench by a perfusable foam ball 12, and the pulsatile output of the heart may be simulated by a cardiac pump 10 (by way of example and not limitation, a Cobe cardiac pump). The cardiac pump 10 may re-circulate saline solution 14. An intracranial bolt 17 may be provided, and in vivo may include tissue contact at the skull and within the ventricle of the brain. As described in detail below, the intracranial bolt 17 may include an intracranial pressure sensor (ICP) sensor 42 and an external ventricular drain 47.

A balun 19 may be used to match impedance of a coax line 24 (for example, the balun may match impedance at 1.8 MHz to the terminus of a 50-ohm coax line such that monitoring will yield a real component Rs of impedance in the range of about 25 to 50 ohms). 1.8 MHz may be employed as the wavelength, which may allow the second arm of the balun 19 to be connected alternatively to various scalp positions to re-direct the orientation of tissue monitoring. However, the method as described herein can be adapted to frequencies, for example, between about 20 KHz and 1 GHz.

By use of the balun 19, tissue impedance at 1.8 MHz may be matched to achieve a standing wave ratio (SWR) less than 1.5 at the end of a three meter segment of 50 ohm coax.

An autotuner 29 may be provided to further tune the resonance of the combined system of coax 24, bolt 15, and tissue 12 to an SWR of less than 1.5. The autotuner may be, for example, activated in a range of about 25-100 mW to optimize the SWR, which may be typically less than 1.5 SWR using a balun. Other tuning methods known to those familiar in the art such as matching transformers, a variety tuning circuits employing capacitors and or inductors, and tuning stubs may also be employed. A balun may be used due to its broadband tuning characteristic, adding flexibility to the structure. Output of the autotuner-reflected power signal component as a monitored voltage may oscillate in a typical systolic-diastolic waveform, relating to the change in impedance due to organ or tissue perfusion. As the SWR is seen to drift over hours, the autotuner 29 may automatically re-tune to regain an optimized SWR at a threshold of less than 1.5 SWR. The new frequency may be data-logged, and may be an indicator of drift of tissue impedance. Further, the re-tuning by the autotuner 29 may optimize perfusional sensitivity. Deterioration of the perfusional waveform may be typically recovered by the autotuning cycle as the threshold of 1.5 SWR is crossed, except in tissue death or loss of adequate heart contractile output threatening death.

The reflected power (RP) component of the SWR from the autotuner 29 may be monitored, and may be indicative of the organ perfusional waveform. A generator 33 of a waveform (by way of example, a 1.8 MHz waveform may be used) may be provided, and the generator 33 may be connected to the autotuner 29. Alternatively, a coax switch may allow the combined system to be measured by a vector analyzer 38, including SWR, Real Loss (RL), and theta (phase shift in degrees at resonance). The coax switch may allow switching the transmitter source to the vector analyzer 38 which limits output power to 10 mW (within acceptable monitoring requirements of power limitation in humans).

The SWR, reflected power, real component of R in the resonant circuit Rs, resonant frequency (theta where all imaginary components of the resonant circuit are minimized), and Q of the circuitry can be monitored continuously. SWR, RL, Rs. and theta all show morphologically identical waveforms consistent with beat-to-beat perfusional change due to organ perfusion by blood. An A/D convertor may allow cross-correlation derivation continuously of the perfusional waveform to the arterial pressure waveform.

The waveform derived from the vector analyzer 38 or the reflected power component of the autotuner 29 may be compared continuously to the somatic intra-arterial perfusional pressure through A/D convertors by the means of cross-correlation. This may provide a measure of lag time between the driving arterial pressure waveform and the organ's perfusional waveform. Further, it may provide a measure of similarity of the two waveforms.

A tomographic reconstruction across a tissue can be created and rendered continuously using SWR, Rs (real component of the complex tissue impedance), RP, RL, or theta (phase angle of imaginary component j of the resonant tissue impedance), all of which have similar waveform and baseline shifts. A tomographic rendering of lag time between perfusional pressure and tissue flow may also be created from such a multiplicity of pairs, and this may be representative of brain stiffness or compliance, which may be an indicator of edema from injury as seen in head trauma, stroke, surgical manipulation, etc.

By way of example, one possible configuration of the electrodes for monitoring a patient's cranium will be described, with reference to specific products and their manufacturers. It will be appreciated that this configuration is given by way of example only, and that various alternate configurations may be possible and other products manufactured by other companies may be used in conjunction with the configurations as described herein. A 4:1 balun (such as that manufactured by Balun Designs, Denton, Tex.) may connect via electrical press-fit junctions to the intracranial bolt and central electrode. The balun may be connected via a 50 ohm coax to an autotuner (such as that manufactured by LDG Electronics Z-11 Proll, St. Leonard, Md.). The autotuner may be connected by switchable input into a transmitter (such as that manufactured by ICOM 7000, Icom America, Bellevue, Wash.) outputting 100 mW at 1.8 MHz. The switchable coax may also connect to a vector analyzer (such as that manufactured by Array Solutions AIM-uhf, Sunnyvale, Tex.).

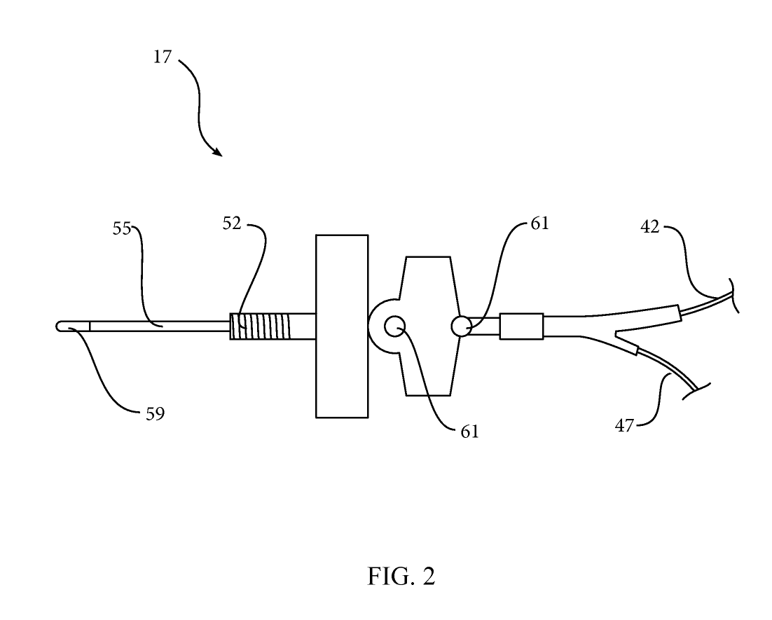

Turning now to FIG. 2, there is shown a view of a possible configuration of an intracranial bolt 17. The intracranial bolt 17 may include a tappable or threaded region 52 for seating into the cranium at the level of the dura. The bolt 17 may be fabricated of electrically conductive material (such as stainless steel or the like). There may be provided an insulated tubular electrode 55 that extends into the ventricle. The insulated tubular electrode 55 may be integral with an external ventricular drain 47, and configured within the intracranial bolt 17. The insulated tubular electrode 55 and external ventricular drain 47 may extend to the level of the lateral ventricle (by way of example, a typical depth may be about 5.5 centimeters below the dura).

The insulated tubular electrode may 55 be insulated by a silicone or Teflon sleeve, except at the exposed tip 59 where it makes electrical contact with brain and cerebrospinal fluid. (By way of example, the tip 59 of the insulated tubular electrode 55 that may be exposed and may have a length of about between 1 and 5 millimeters.) The lumen of the insulated tubular electrode 55 may also contain an inserted, electrically separated pressure transducer, or ICP monitor, 42 to provide continuous ICP monitoring from within the cerebrospinal fluid-containing cavity of the lateral ventricle. The components of the installed bolt outside of the scalp, such as the electrical contacts 61 for contact to a balun, may allow direct electrical connection to circuitry for monitoring of tissue impedance between the cranium and catheter tip, or exposed tip, 59 within the brain. Impedance may be monitored between the exposed tip 59 of the electrode 55 and the tapped bolt.

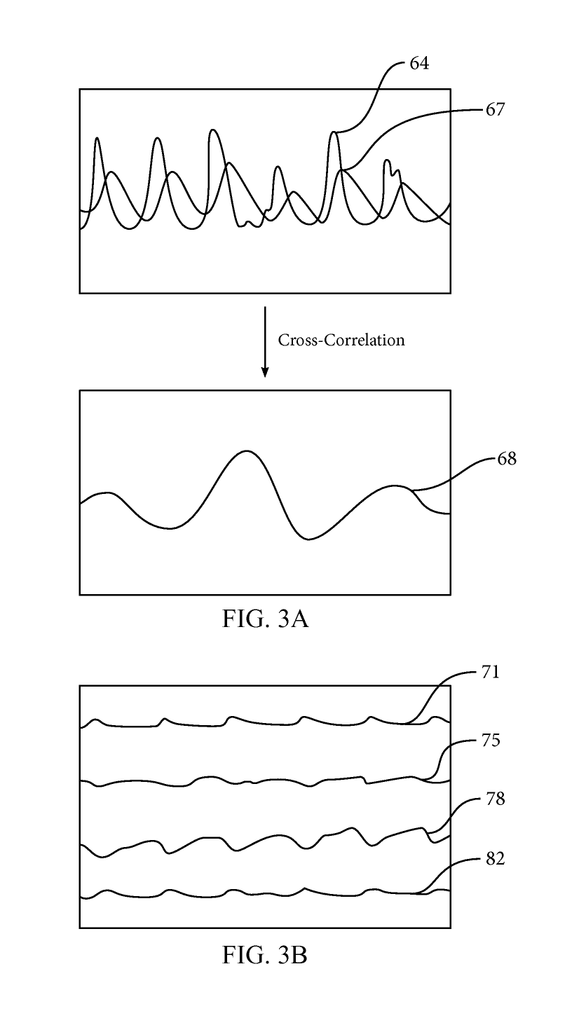

Turning now to FIG. 3A, there is shown an illustrative graphical representation of an arterial pressure pulse and the RP component of SWR derived from the brain. The peak 64 of the bloodflow waveform lags behind the peak 67 of the ICP waveform. The lag time of flow, represented by RP, can be quantified by the statistical method of cross-correlation between waveforms, and the subsequent cross-correlation 68 is shown. This rendering may be typically performed continuously during acquisition of waveforms. A measure of waveform similarity may also be indicative of brain stiffness or compliance. FIG. 3B shows plots from the vector analyzer of SWR, theta, RL, and Rs. The SWR plot 71, Z-magnitude of impedance 75, phase angle 78, and RP, (reflected power loss components of SWR) 82, all show pulsatility in FIG. 3B. Pulsatility may be seen in all waveforms, consistent with the beat-to-beat alteration of impedance in the tissue as monitored at 1.8 MHz. It may be noted that the lowest SWR may never be at 1.8 MHz, though it may be near 1.8 MHz, due to tuning. Further, the resonant frequency may never be at the point of lowest SWR. Greatest sensitivity to pulsatile change may be seen at the resonant point.

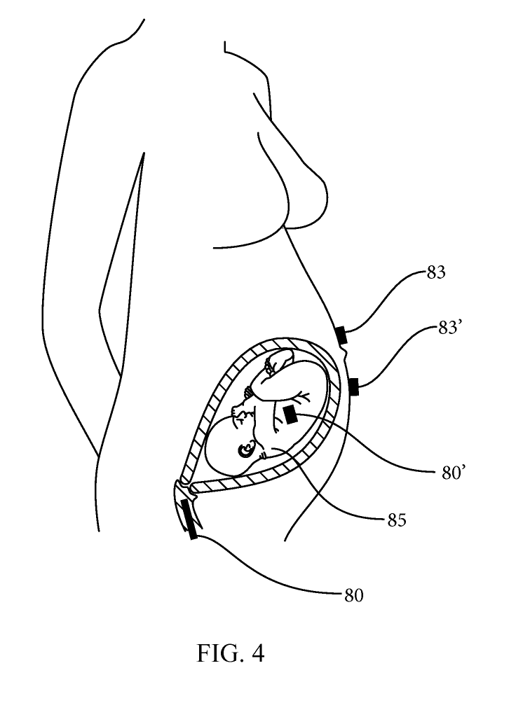

Turning now to FIG. 4, there is shown another example of an electrode configuration as disclosed herein. This configuration involves the monitoring of a developing fetus. From 24 weeks to term gestation at 40 weeks, fetal heart rate (FHR) may be a valuable indicator of fetal well-being. Specifically, FHR and fetal heart rate variability may signal a threatening change or a recovered or seemingly optimized condition. A long-term, wearable, data-logging apparatus which may signal to the mother and obstetrician a condition of fetal well-being, may be achieved by means of placement of a central electrode within the natural orifice of the vagina and a further electrode which may be placed upon the mother's skin straddling the intervening fetus within the gravid uterus.

FIG. 4 shows the sagittal cross-sectional placement of a central vaginal electrode 80 beneath the cervix and near the fetal head. The central electrode 80 may be configured as a conductive, expandable tampon to maintain electrical contact with the high wall below the cervix of the vagina. A second abdominal electrode 83 may adhere to the maternal skin above the dome of the uterus. The intervening tissue may include the fetus 85. The heart and fetal somatic pulsation due to perfusion of the fetus 85 may alter the resonant impedance, and may allow derivation of a fetal heart rate. Another alternate configuration, which may be utilized after rupture of membranes during parturition, may include a central electrode 80′ placed within the amniotic sac as shown. A second electrode 83′ may be positioned on the maternal abdomen or within the vagina, to “see” across the fetus 85, i.e., the fetus 85 would be part of the intervening tissue between the central electrode 80′ and the second electrode 83′.

While a tomographic reconstruction of the maternal abdomen may be achieved, this may be of less practical interest than the tissue which changes beat-to-beat due to the electrically resonant path of very low current through the fetus. The electrode pair (consisting of the central electrode 80 or 80′ and the second abdominal electrode 83 or 83′) may be similarly connected via a balun (by way of example, a 9:1 balun) to achieve tunable resonance in the SWR range of less than 2:1. Identical variables of SWR, RL, Rs, and theta carry the FHR pattern in addition to maternal heart rate, maternal breathing rate, and fetal movement. The fetal heart rate may be digitally filtered and data-logged against these other changing variables by a post-processing computer. The continuous monitoring can be wirelessly transmitted by well-familiar interfaces known in the art, such as Bluetooth, cellular phone connections, etc.

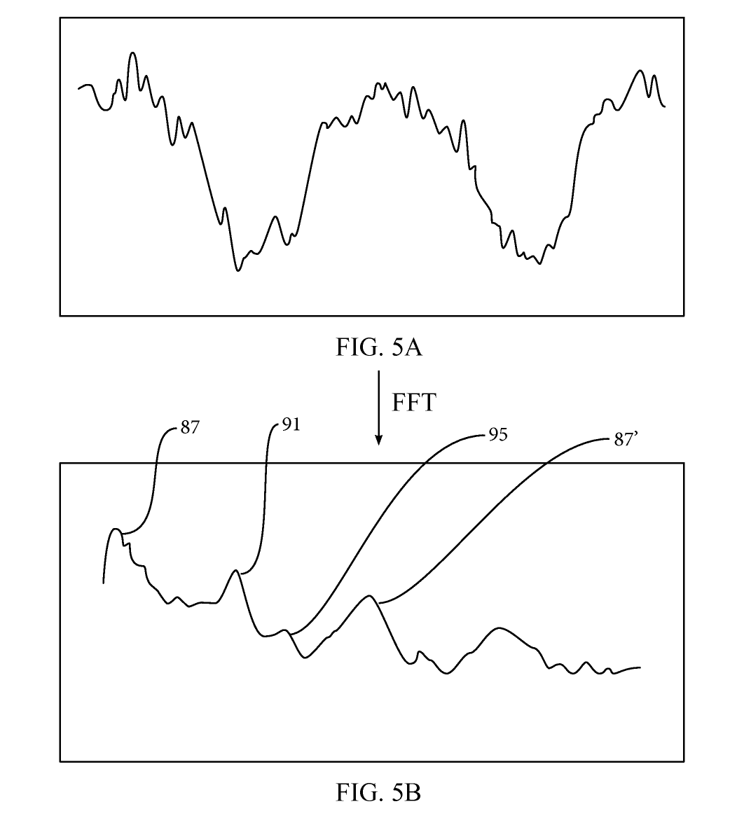

FIG. 5A shows a graphical representation of derived impedance as RP of the SWR component from a pregnant mother. The maternal breathing rate (large, slow oscillations), the maternal heart rate (prominent, fast oscillations), and the fetal very fast heart rate on top of the maternal heart rate are seen. FIG. 5B shows a Fast Fourier Transform (FFT) of the RP of the SWR component of the resonant impedance tracing. The maternal breathing rate 87 may be seen, as well as the material heart rate 91, and fetal heart rate 95. The first harmonic 87′ of the maternal heat rate may also be seen. Variability is reflected in the width of the bins in the FFT.

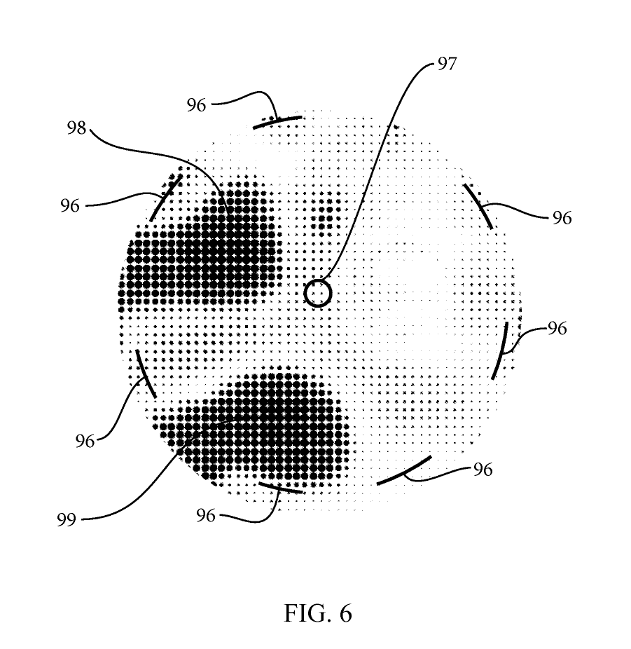

FIG. 6 is a cross-sectional tomogram of tissue lesioning by pH shift using an array of 7 circumferential electrodes 96 and a central electrode 97. pH shift increases ionic conductivity about an electrode by cathode OH-tissue deposition and anodic H+ ion deposition. In FIG. 6, shifted pH is represented by darker shading. The shifted pH is visible surrounding the cathode 98 and the anode 99.

Other anatomical regions, such as those mentioned above, may be used in conjunction with the present disclosure. The present disclosure may give the ability to monitor continuously between a pair of electrodes so configured, or among an array of electrodes so configured for tomographic rendering. Certain procedures are known to change tissue impedance. These may include tissue ablation (destruction) thermally by a variety of heating methods or chemically. A specific example may be destruction of prostatic glandular tissue within the capsule of the organ to diminish external compressional obstruction upon the intraglandular urethra. Another example may be lesioning of metastases in solid organs.

Further, tissues may be improved in functionality by thermoplasty, a method of shrinking tissue, specifically collagen, or weakening or strengthening tissue planes. Examples may include the external urethral sphincter for incontinence, the functionally abnormal reactivity of smooth muscle of the bronchus for asthma, and chronic obstructive pulmonary disease, as well as ligaments of a joint capsule which have become lax.

Yet a further example may be the focal delivery of a chemotherapeutic agent as a drug or even a pH shift to achieve selected, controlled tissue injury or killing, for example neoplasm. A yet further example may the monitoring of a disease process where inflammation and edema are treated by chemotherapeutic or radiofrequency means, for example pneumonitis. In these instances, the shift or resonant frequency in the current pathway between electrode pairs (or configurations of multiple electrodes) may represent a change in conductivity with alteration of impedance. Paired pole monitoring as described herein or a tomographic array of such resonance may be employed to monitor the treatment process.

It will be readily apparent to one having skill in the art that adaptations of these concepts can be extended to a variety of tissues with both natural orifices or physician-created paths into tissues. Further, the interrogating frequencies to bring out properties of tissue under surveillance may extend beyond pulsatile perfusion due to blood. It can thus be seen that such methods are within the scope of this patent.

A system for monitoring the status of an internal tissue is described herein, the system comprising: at least one central electrode configured to be placed adjacent to at least a portion of the internal tissue at least one second electrode configured to be placed proximate to the at least one central electrode to generate a resonant circuit across the internal tissue. The resonant circuit may include an autotuner and a frequency source. The resonant circuit may also include a coax line. The resonant circuit may further include an impedance matching technique, and the impedance matching technique may comprise a balun.

The resonant circuit may include a vector analyzer. The central electrode may be configured to be placed within a natural orifice, or may be configured to be placed within a created pathway into the substance of a tissue or organ. The resonant circuit may have a resonance between about 20 KHz and 1 GHz. The resonant circuit may have a resonance near 1.8 MHz according to one configuration. The at least one second electrode may comprise an array of electrodes.

A system for creating a resonant circuit may include: a frequency source, an autotuner, a coax line, a balun, an internal electrode configured to be placed internally, and a second electrode configured to be placed externally. The system may be configured to measure resonance across an intervening tissue extending between the internal electrode and the second electrode.

A system for creating a resonant circuit is disclosed herein, wherein the resonant circuit may include components, the components including a frequency source, an autotuner, a coax line, a balun, an internal electrode configured to be placed internally, and a second electrode configured to be placed externally. The system may further include the component of an intervening tissue extending between the internal electrode and the second electrode, and the only changing component of the resonant circuit may be the intervening tissue extending between the internal electrode and the second electrode.

A method for creating a resonant circuit within and across tissue is disclosed, the method comprising: inserting a central electrode within an orifice or a created pathway in tissue, placing a second electrode within or upon the tissue, and generating an electrical signal between the central electrode and the second electrode to create a resonant circuit comprising the tissue. The resonant circuit may include a balun, a coax line, and auto tuner, and a frequency source. The method may include continuously measuring a resonant impedance. The method may include utilizing the resonant impedance to guide thermoplasty or chemoplasty of a tissue.

The method may include using a vector analyzer to plot SWR, theta, RL, and/or Rs. Likewise the SWR, Z-magnitude of impedance, phase angle, and RP, (reflected power loss components of SWR) can be plotted and used to all show pulsatility in the tissue being monitored.

There is thus disclosed an improved electrode configuration and method of use. It will be appreciated that numerous changes may be made to the present invention without departing from the scope of the claims.

What is claimed is:

1. A system for monitoring the status of an internal tissue, the system comprising:
an internal electrode configured to be placed adjacent to at least a portion of the internal tissue; and
at least one second electrode configured to be placed proximate to the internal electrode to generate a resonant circuit across the internal tissue; and
at least one frequency generator disposed in communication with at least one of the internal electrode and the at least one second electrode for creating a resonant circuit which passes through the internal electrode and the at least one second electrode and tissue therebetween.

2. The system of claim 1, wherein the resonant circuit further includes a balun disposed in electrical communication with the internal electrode and the at least one second electrode.

3. The system of claim 1, wherein the resonant circuit includes an autotuner.

4. The system of claim 3, wherein the autotuner is connected to the balun by a coax line.

5. The system of claim 4, further comprising a vector analyzer disposed in communication with the autotuner.

6. The system of claim 1, wherein the resonant circuit further includes a vector analyzer.

7. The system of claim 6, wherein the vector analyzer is configured to monitor at least one of standing wave ratio, reflected power, real component of R in the resonant circuit Rs, resonant frequency (theta where all imaginary components of the resonant circuit are minimized), and Q of the circuitry.

8. The system of claim 1, wherein the resonant circuit has a resonance between about 20 KHz and 1 GHz.

9. The system of claim 1, wherein the resonant circuit has a resonance near 1.8 MHz.

10. The system of claim 1, wherein the at least one second electrode comprises an array of electrodes.

11. A system for creating a resonant circuit, the system including:
a frequency source, an autotuner, a coax line, a balun, an internal electrode configured to be placed internally, and a second electrode configured to be placed externally disposed in electrical communication with the frequency source.

12. The system of claim 11, wherein the system is configured to measure resonance across an intervening tissue extending between the internal electrode and the second electrode.

13. A method for monitoring tissue, the method comprising:
disposing an internal electrode on one side of a tissue to be monitored,
placing a second electrode within or upon the tissue so that a part of the tissue is disposed between the internal electrode and the second electrode, and
generating an electrical signal between the internal electrode and the second electrode to create a resonant circuit comprising the tissue.

14. The method according to claim 13, wherein the resonant circuit includes a balun, a coax line, and auto tuner, and a frequency source.

15. The method according to claim 13, wherein the method further comprises continuously measuring a resonant impedance.

16. The method according to claim 15, wherein the method further comprises utilizing the resonant impedance to guide thermoplasty or chemoplasty of a tissue.

17. The method according to claim 13, wherein the method comprises monitoring the electrical signal by monitoring at least one of standing wave ratio, reflected power, real component of R in the resonant circuit Rs, resonant frequency (theta where all imaginary components of the resonant circuit are minimized), and Q of the circuitry, to thereby monitor perfusion of fluid through the tissue.

18. The method according to claim 17, wherein the method comprises using a vector analyzer to monitor the electrical signal.

19. The method according to claim 13, wherein the electrical signal has a frequency and wherein the method comprises adjusting the frequency of the electrical signal to maintain a resonant circuit through the tissue.

20. The method according to claim 19, wherein the method comprises using an autotuner to maintain the resonant circuit through the tissue using autotuner-reflected power signal output in a systolic-diastolic waveform to determine change in impedance due to tissue perfusion.
